(19) United States
(12) Patent Application Publication
(10) Pub. No.: US 2006/0155296 A1
(43) Pub. Date: Jul. 13, 2006

(54) Three-dimensional implantable bone support

(75) Inventor: Goetz Martin Richter, Gaiberg (DE)

Correspondence Address: FLASTER/GREENBERG P.C.;8 PENN CENTER, 1628 JOHN F. KENNEDY BLVD., 15TH FLOOR, PHILADELPHIA, PA, 19103, US

(73) Assignee: Celonova BioSciences, Inc., Newnan, GA (US)

(21) Appl. No.: 11/328,345

(22) Filed: Jan. 9, 2006

Related U.S. Application Data
(60) Provisional application No. 60/641,968, filed on Jan. 7, 2005.

(51) Int. Cl. A61F 2/34

(52) U.S. Cl. 606/94

(57) ABSTRACT

The present invention relates to an expandable semi-compliant device that may be used for the treatment of diseased or injured bone tissues, and a method of using the same. The semi-compliant device is inserted into the interior space of a cancellous bone tissue, and is filled with a suitable material to provide internal structural support to the bone. The semi-compliant device may also act as a carrier for medicinal, radiological, or thermal treatments of the diseased bone.

### CROSS-REFERENCE TO RELATED APPLICATIONS

This application claims the benefit under 35 U.S.C. § 119(e) of U.S. Provisional Patent Application No. 60/641,968, filed Jan. 7, 2005, the disclosure of which is incorporated herein by reference.

### BACKGROUND OF THE INVENTION

There is presently known in the art a wide range of treatments for diseased or injured cancellous bone tissues in mammals. Cancellous, or spongy bone, has a trabecular (honeycomb structure) and a high level of porosity relative to cortical bone. The spaces between the trabeculae are filled with red bone marrow containing the blood vessels that nourish spongy bone. Spongy bone is found in bones of the pelvis, ribs, breastbone, vertebrae, skull, and at the ends of the arm and leg bones.

All bones are subject to damage by trauma, disease processes, or fractures, such as, but not limited to, osteoporosis, osteoporotic bone, osteoporotic fractured metaphyseal and epiphyseal bone, osteoporotic vertebral bodies, fractured osteoporotic vertebral bodies, fractures of vertebral bodies due to tumors, especially round cell tumors, avascular necrosis of the epiphyses of long bones, especially avascular necrosis of the proximal femur, distal femur and proximal humerus, defects arising from endocrine conditions, and metastatic tumors. The bones comprising the vertebral spine are particularly difficult to treat due to the complexity of their anatomical structure. Effective treatment of the vertebra is further exacerbated by the proximity of the spinal cord to the nerves emanating therefrom.

Two minimally invasive procedures that have gained popularity in the treatment of fractured or diseased bones, and in particular the vertebra, are percutaneous vertebroplasty and Kyphoplasty. U.S. Pat. No. 6,273,916 describes a method and apparatus for performing vertebroplasty. Vertebroplasty is a procedure wherein a cement-like material, such as polymethylmethacrylate (“PMMA”), is injected under high pressure directly into the vertebral cavity. The cement-like material is permitted to cure, and upon hardening, provides structural support to the affected vertebra.

In Kyphoplasty, a small incision is made in the back. Using fluoroscopic imaging techniques, a surgeon guides a cannula to a desired position, inserts a drill through the cannula, and bores through the cortical wall into the cancellous bone to define a channel within the vertebral body. The drill is removed and a balloon catheter is inserted into the channel. The balloon catheter is then inflated to compress the cancellous bone against the inner cortical wall to define a cavity therein. A particular advantage of this procedure for compression fractures is that inflation of the balloon catheter restores a portion of the vertebral height. Following deflation and removal of the balloon catheter, a cement-like material, such as that used in vertebroplasty, is injected to fill the cavity. The cement is permitted to cure, and the surgical site is closed.

Variations of percutaneous vertebroplasty and Kyphoplasty are known in the prior art. For example, U.S. Pat. No. 5,827,289 discloses using a balloon to form or enlarge a cavity or passage in a bone, especially in, but not limited to, vertebral bodies and to deliver therapeutic substances to bone in an improved way. U.S. Pat. No. 6,632,235 discloses using inflatable devices for reducing fractures in bone and treating the spine. U.S. Patent Application Publication No. US 2003-0050644 A1 discloses employing an expandable body that is inserted into bone over a guide wire. U.S. Patent Application Publication No. US 2005-0234456 A1 discloses using an implantable medical device for supporting a structure. U.S. Pat. No. 6,348,055 discloses using a conduit for delivering an implant material from a high pressure applicator to an implant delivery device. U.S. Pat. No. 6,033,411 discloses using precision depth-guided instruments to perform percutaneous implantation of hard tissue implant materials.

While the aforementioned procedures represent significant advances in the treatment of bone injuries and diseases, they are not without risk. A risk common to both procedures is the exfiltration of the cement from a fracture site in the treated bone. While these risks are more pronounced in vertebroplasty, due to the high injection pressures, exfiltration of the cement from the fracture site can lead to thrombosis, spinal stenosis, or nerve root compression, and in rare cases pulmonary embolus.

A further limitation of the aforementioned procedures is that once the bone cement has cured, subsequent removal of the cement from the vertebral body is prohibitive, particularly in the case of vertebra in the spine.

Similarly, the aforementioned methods are reparative and make no provision for the treatment of any underlying disease condition which may have caused or contributed to the fractures necessitating the application of these methods in the first place.

Accordingly, despite these recent advances in the art, there remains a continuing need for improved devices and methods for treating bone fractures and disease conditions.

### BRIEF SUMMARY OF THE INVENTION

The present invention is directed to a method of treating diseased or injured bone tissue comprising selecting an interior area in a bone tissue to be treated, inserting a device into the interior area of the bone tissue to be treated, and internally supporting the bone tissue using the device during treatment.

The present invention is also directed to a device for treating diseased or injured bone comprising a catheter, wherein the catheter comprises a main body defining at least one interior passage therethrough, an expandable semi-compliant structure, wherein the semi-compliant structure defines an interior space, and a removable fastener, wherein the fastener releasably connects the catheter to the semi-compliant structure.

### BRIEF DESCRIPTION OF THE SEVERAL VIEWS OF THE DRAWINGS

FIG. 1 is a side elevational view of a detachable semi-compliant structure and catheter according to one embodiment of the invention inserted into a cavity defined in the cancellous bone of a vertebra.

FIG. 2 is a side elevational view of the detachable semi-compliant structure and catheter of FIG. 1 as expanded by a bone supporting material.

FIG. 3 is a side elevational view of decoupling of the catheter of FIG. 1 from the semi-compliant structure of FIG. 1 and sealing of the semi-compliant structure.

FIG. 4 is a side elevational view of a semi-compliant structure after implantation in a vertebra.

FIG. 5 is a transverse cross-sectional view of a spinal vertebra and arthroscopic probe inserted therein.

FIG. 6 is a transverse cross-sectional view of a spinal vertebra having a semi-compliant structure and arthroscopic probe that is partially inserted into a vertebral body.

FIG. 7 is a transverse cross-sectional view of a spinal vertebra having a semi-compliant structure and arthroscopic probe that is fully inserted into a vertebral body.

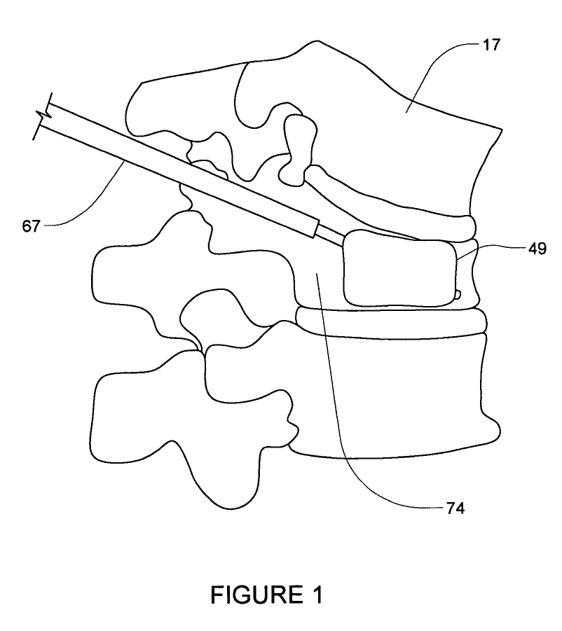

### DETAILED DESCRIPTION

The present invention relates to the field of orthopedic surgical devices and techniques. The method of treatment of the present invention involves using a catheter 67 that is connected to a preferably detachable semi-compliant structure by a removable fastener, preferably a screw device. The fastener releasably connects the catheter 67 to the semi-compliant structure 49 and is capable of coupling the semi-compliant device 49 to the catheter 67 and decoupling the semi-compliant device 49 from the catheter 67.

The catheter 67 has a main body defining at least one interior passage therethrough, the semi-compliant structure defines an interior space, and the semi-compliant structure comprises a sealable port that allows for communication between the interior passage of the catheter and the interior space of the semi-compliant structure.

“Semi-compliant structure” is defined herein as a malleable, expandable, non-rigid structure. This is in contrast to a totally compliant structure, or a rigid, non-compliant structure. Semi-compliant structure more specifically defined as a structure that has a specific compliance rate of about 10% to about 30%. It should be understood that such rate is non-limiting to the scope of the invention. The compliance rate of the semi-compliant structure is defined as the rate at which the structure yields to pressure or force without disruption, or an expression of the measure of the ability to do so, such as an expression of the distensibility of the semi-compliant structure, in terms of unit of volume change per unit of pressure change, when it is filled with liquids or other materials.

The semi-compliant structure may be temporarily or permanently inserted in an interior area such as a cavity or other space within diseased or injured cancellous bone tissue of a mammal in order to internally support the bone and/or to treat such diseases or injuries, and to alleviate symptoms of such diseases or injuries, such as back pain. The detachable semi-compliant structure expands upon introduction, typically by injection, of a suitable bone supporting material, through a passage within the catheter, and the semi-compliant structure provides containment and maintenance of the bone supporting material therein. The detachable semi-compliant structure is preferably shaped such that upon expansion, the structure will generally adapt and conform three-dimensionally to the dimensions of the exterior area such as a cavity defined within the internal cortical walls of the bone to be treated. The detachable semi-compliant structure prevents the exfiltration of the bone supporting material from the fracture site through use of a preferred semi-permeable membrane, and facilitates controlled drainage from the structure, thereby avoiding the deleterious effects described herein above.

To provide additional containment and maintenance of the bone supporting material within the structure, the structure may be provided with a sealable port, through which the catheter communicates with the semi-compliant structure. The port may be sealed upon detachment of the catheter to prevent the bone supporting material from exuding from within the structure. This arrangement further facilitates pressurized containment and maintenance of the bone supporting material within the structure. The port may remain open, but where the bone supporting material hardens and so cannot exude from the port. In another embodiment, the port may be temporarily sealed so that the catheter can be reattached to the port, and the bone supporting material can be removed as necessary.

The semi-compliant structure may be formed from any suitable biocompatible material that is malleable and durable, such as, but not limited to, stainless steel, titanium, polymers such as, for example, polymeric materials and plastics such as polyester and polyethylene, polylactic acid and copolymers of these polymers with each other and with other monomers, resorbable synthetic materials such as, for example, suture material, Nitinol, or any other suitable material as known to those of skill in the art, including combinations of such materials. The suitable biocompatible material is preferably in the form of a thin metallic film material that is super-elastic and possesses excellent rubber-like shape retention. Nitinol, a metal alloy of nickel and titanium, is a particularly suitable biocompatible material because Nitinol has the ability to withstand the corrosive effects of biologic environments, such as that inside cancellous bone tissue. In addition, Nitinol also has excellent wear resistance and shows minimal elevations of nickel in the tissues in contact with nitinol. Betz et al., Spine, 28(20S) Supplement:S255-S265 (Oct. 15, 2003). The use of a suitable Nitinol as a preferred biocompatible material in implantable balloons is disclosed in U.S. Pat. No. 6,733,513, which is incorporated herein by reference.

The semi-compliant structure is preferably in the form of an expandable three-dimensional balloon. Where the semi-compliant structure is permanently inserted into cancellous bone tissue, the biocompatible material of the structure is made of a suitable surface material, such as, but not limited to those mentioned above, to provide a bone-friendly membrane for incorporation and healing and to help improve or accelerate the attraction of healthy bone cells.

In applications where disease is the underlying cause of the bone fracture, an object of the present invention further contemplates that the semi-compliant structure serve as a carrier for a treatment for a disease or injury. The invention contemplated herein includes medicinal, radiological and thermal treatments for the underlying disease conditions. Such medical treatments may include, but are not limited to, such treatments comprising drugs such as, but not limited to, Cisplatin, Taxol™, Adriamycin™, Doxorubicin, Melphalan, Cyclophosphamide, Carboplatin, Methotrexate, or similar treatments known to those in the art for treating bone diseases. Such radiological treatments include, but are not limited to, radiation therapy which can be used for treatment of malignant bone disease to prevent further fractures and pain, or interventional procedures which can be applied to malignant bone disease by means of embolization (transvascular occlusion).

The bone supporting material may include a number of materials that are selected based on the purpose of the treatment. Where the treatment encompasses permanent bone support, the bone supporting material includes bone cement that may be injected as a liquid and then which hardens within a short period of time. Where the treatment encompasses temporary support of the bone, the bone supporting material may be injected as a liquid, and will remain a liquid form during the time required for support. It can then be readily withdrawn when the treatment procedure is complete and/or replaced if additional treatment is needed. In alternative embodiments, the bone supporting material may be in the form of a pliable gel-like material to provide support and energy attenuation for the bone structure.

As may be seen in reference to the various drawings, the present invention includes a catheter 67 having at least one lumen or other long extending passage way, preferably a multi-lumen catheter 67, with a detachable semi-compliant structure 49 for temporary or permanent placement in a cavity 74 defined in bone tissue such as cancellous bone tissue 17. The present invention further comprises methods of treating bones which have been fractured through trauma or through disease processes, such as, but not limited to, osteoporosis, osteoporotic fractured metaphyseal and epiphyseal bone, osteoporotic vertebral bodies, fractures of vertebral bodies due to tumors, especially round cell tumors, avascular necrosis of the epiphyses of long bones, especially avascular necrosis of the proximal femur, distal femur and proximal humerus and defects arising from endocrine conditions, metastatic tumors, long bone (i.e., traumatic or spontaneous bone fractures or other local distortions of bone structures), such as cervical, thoracic, lumbar, and sacral fractures, and the like.

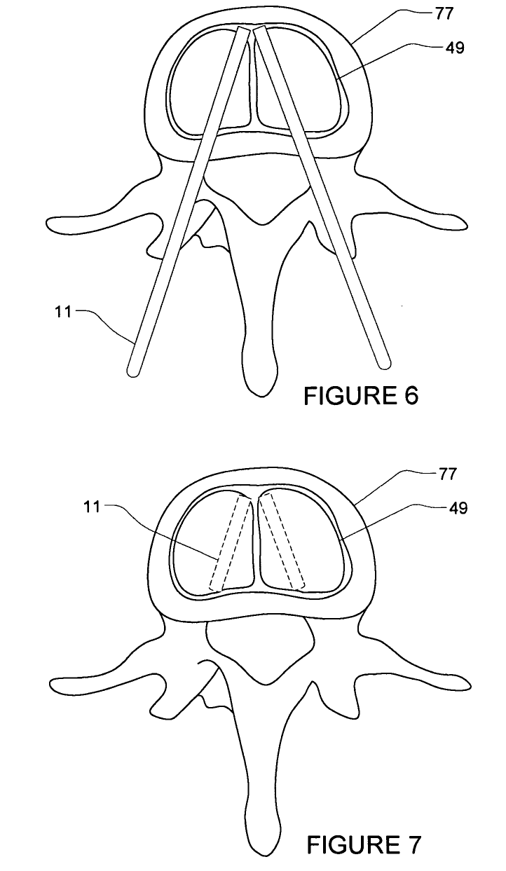

The detachable semi-compliant structure 49, as best shown in FIGS. 6 and 7, is shaped such that it generally conforms to dimensions of a cavity 74 selected within the internal cortical walls of the cancellous bone tissue 17. The cavity 74 may be simply identified and/or defined within the internal cortical walls by any suitable procedure familiar to those of skill in the art, such as, but not limited to, drilling, insertion of a precursor inflatable device, and other related methods. The dimensions of the cavity 74 may be predetermined using minimally invasive image-guided techniques such as, but not limited to, X-ray, CT scan or intraoperative CT imaging, ultrasound, computed tomography, MR/CT image registration, three dimensional visualization, optical localization, and magnetic resonance imaging (MRI), or any other suitable imaging techniques. Preferably, the walls of the semi-compliant structure 49 have a compliance rate of about 10% to about 30%, to provide engagement of the structure with the cavity 74 walls comprising either cancellous bone 17 or the internal walls of the cortical bone.

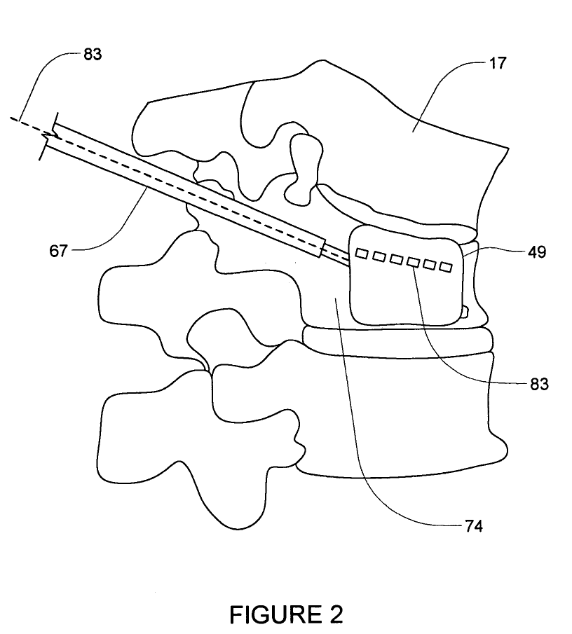

As depicted in FIG. 2, the detachable semi-compliant structure 49 is expandable upon injection of a suitable bone supporting material 83 through a lumen of the multi-lumen catheter 67, with the structure 49 providing containment and maintenance of the bone supporting material 83 therein and additional structural support to the cancellous bone tissue 17. The characteristics of the bone supporting material 83 are selected based upon whether the structure 49 will be a permanent implantation or whether the structure 49 will be temporarily implanted for a sufficient duration to permit a bone fracture to heal.

For permanent implant treatments, the bone supporting material 83 may be a cement-like material made of a formulation known or to be developed in the art, such as those based on polymethylmethacrylate (“PMMA”), or other suitable biomaterial alternatives or combinations, including, but not limited to, dextrans, polyethylene, carbon fibers, polyvinyl alcohol (PVA), or poly(ethylene terephthalate) (PET), such as those used in conventional vertebroplasty or Kypohplasty procedures. More preferably, the cement-like material is PMMA. Specific formulations of PMMA are known in the art and are commonly used in bone implants. Such formulations include, but are not limited to those disclosed in, for example, U.S. Pat. Nos. 4,526,909 and 6,544,324, which are incorporated herein by reference.

One of the primary objects of the present invention is to prevent exfiltration of the cement-like material from the fracture site and its resulting physiological risks. This prevention is possible due to the containment and maintenance of the cement-like material within the semi-compliant structure 49.

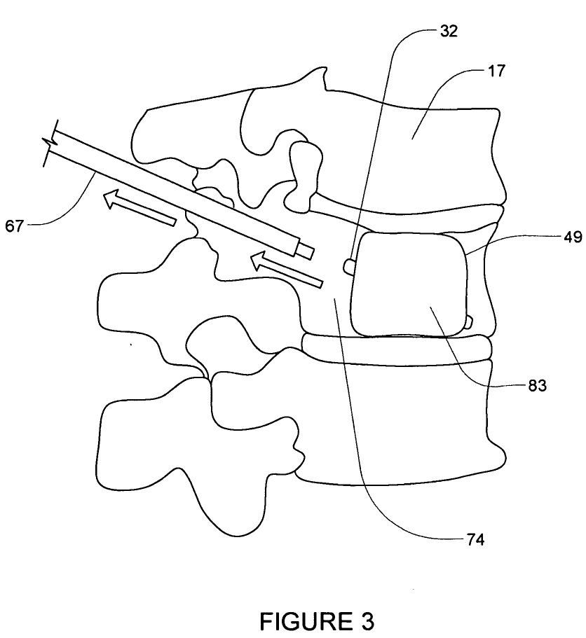

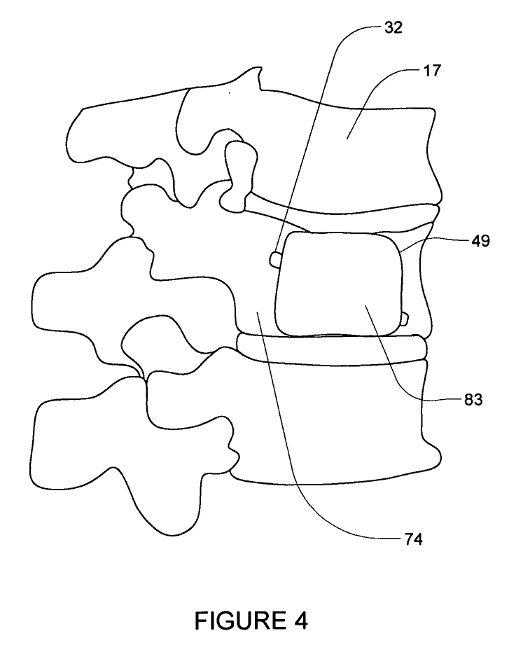

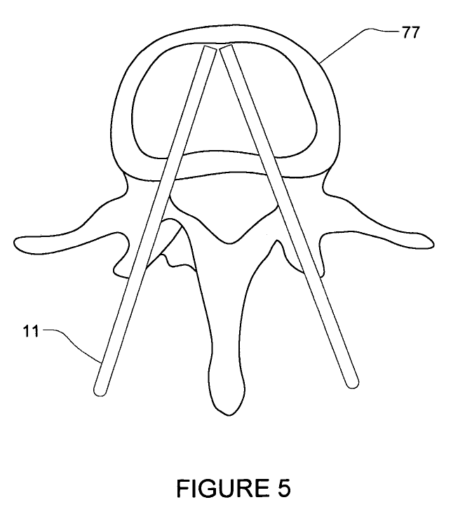

To provide additional containment and maintenance of the bone supporting material 83 within the semi-compliant structure 49, the structure 49 may be provided with a sealable port 32, as shown in FIGS. 3 and 4, through which the catheter 67 communicates with the semi-compliant structure 49. The port 32 may be sealed upon detachment of the catheter 67 to prevent the bone supporting material 83 from leaching out of the structure 49. This arrangement further facilitates pressurized containment and maintenance of the bone supporting material 83 within the structure 49. Additionally, a sealable port 32 also prevents the infiltration of biologic fluids into the semi-compliant structure 49, thereby improving the structure's durability by preventing corrosion and degradation of the walls of the internal semi-compliant structure 49. Alternatively, the catheter 67 may be left attached to the semi-compliant structure 49 until such time as the bone supporting material 83 has cured. Such curing time generally takes about 2 to about 10 minutes if PMMA is used as the bone cement. Once the PMMA has cured, the catheter 67 may then be detached with minimal risk of the material leaching from the sealable port 32, as shown in FIG. 3. The reverse arrows in FIG. 3 from the bone tissue 17 indicate the direction in which the catheter 67 moves after injection of the bone supporting material 83 into the semi-compliant structure 49 and decoupling therefrom. However, because this process potentially leaves the structure 49 temporarily open, care should be taken to the extent necessary, to avoid infiltration of the biological fluids into the structure 49.

The device of the present invention may also be utilized for temporary implantation in cancellous bone 17, potentially offering a more advantageous bone setting technique compared to contemporary procedures which rely on insertion of metallic rods, pins or screws to maintain a bone's structure while the fracture is permitted to heal. In this instance the semi-compliant structure 49 would likely require a port having a valve to maintain the strength and rigidity of the structure while the fracture heals, but to allow access to the bone supporting material 83 for evacuation at a later time. In this instance the sealable port 32 also provides for reattachment of the catheter 67 to permit removal of the bone supporting material 83 and extrication of the structure from the bone 17.

The characteristics of the bone supporting material 83 are selected such that it assumes a rigid or semi-rigid state while the bone is healing and is capable of being dissolved, melted, or otherwise withdrawn from the semi-compliant structure 49 once the healing processes have progressed to a point where internal support is no longer necessary. Once the bone supporting material 83 is evacuated from the semi-compliant structure 49, the structure 49 may then be extricated from the bone to permit final healing of the bone 17. An advantage of the semi-compliant structure 49 over that of metallic rods or pins is that its compliance will facilitate its removal with minimal trauma to the cancellous bone 17 as it is extricated.

The semi-compliant structure 49 may be formed from any suitable biocompatible material, such as, but not limited to, stainless steel, titanium, polymers such as, for example, polymeric materials and plastics such as polyester and polyethylene, polylactic acid and copolymers of these polymers with each other and with other monomers, resorbable synthetic materials such as, for example, suture material, Nitinol, or any other suitable material as known to those of skill in the art, including combinations of such materials. Preferably, the semi-compliant structure 49 will be formed from a biocompatible metallic film material, appropriately shaped to generally conform or adapt to a cavity 74 defined in the internal structure of the bone 17 selected for treatment. An alloy of Nickel and Titanium, commonly known as Nitinol, is well suited to this application, as a result of its proven biocompatibility and its ability to withstand the corrosive effects of biologic environments. Other desirable properties for the metallic film material, and Nitinol in particular, are its super-elasticity and shape memory, which facilitates insertion of the catheter 67 into the cavity 74 defined in the cancellous bone 17. Moreover, Nitinol's stress-strain characteristics make it an excellent choice to provide additional structural support to the bone 17 in combination with the bone supporting material 83.

For bone treatments encompassing permanent placement of the structure 49, the biocompatible material is provided with a suitable surface treatment to provide a bone-friendly matrix for incorporation and healing within the cancellous bone 17. In applications where implantation of the structure will be a temporary restorative measure, the surface is prepared to avoid incorporation of and to reduce the adhesion of cancellous bone 17 to the semi-compliant structure 49 thereby facilitating extrication and minimizing trauma to the cancellous bone 17.

Due to the wide range of applications for the semi-compliant structure 49, the bone supporting material 83 may include a number of materials that are selected based on the underlying purpose of the treatment. Where the treatment is for permanent bone support, the bone supporting material 83 includes a cement-like material, such as the previously described PMMA formulation, that may be injected as a liquid, paste or gel, and then permitted to cure or harden within a short period of time. Because the cement-like material is contained and maintained within the semi-compliant structure 49, a wider range of cement-like materials is possible, as the material would not encounter the same biochemical environment as faced by uncontained applications.

In instances where the treatment is for the temporary support of the bone 17, the bone supporting material 83 is injected as a liquid, remains a liquid during the time required for support, and then can be readily withdrawn when the procedure has been completed. In alternative embodiments, the bone supporting material 83 may be in the form of a pliable gel-like material to provide support and energy attenuation for the bone structure.

In applications where disease is a contributing or underlying cause of the bone fracture, a further object of the present invention contemplates that the semi-compliant structure 49 serves as a carrier for treatment of the disease. The aspects of the invention contemplated herein include medicinal, radiological or thermal treatments for the underlying disease condition.

In cases of medicinal treatment regimens, the surface of the metallic film material may be impregnated or coated with a time-release medication targeting the specific disease condition from within the bone itself. Alternatively, the medication may be diffused through a semi-permeable biocompatible material selected for the structure 49 to treat a disease or injury of the bone 17.

In the case of radiological treatment, the radiological treatment is admixed with the bone supporting material 83 by introducing the admixture into the semi-compliant structure 49, such that it is contained and maintained within the semi-compliant structure 49. In this case, the radiological treatment could be withdrawn from the semi-compliant structure 49, after the appropriate exposure to cancellous bone tissue 17 has been attained. Moreover, as the present invention contemplates temporary implantation of the structure 49, it may also be replaced during radiological treatments or after the completion of all radiological procedures.

The thermal treatment may be provided in the first instance as the bone supporting material 83 is introduced into the semi-compliant structure 49. The temperature of the bone supporting material 83 may be adjusted to a desired level prior to introduction into the semi-compliant structure 49. Alternatively, the appropriate temperature may be attained by catalytic reaction of the selected bone supporting material 83. Re-treatment of the bone tissue 17 may be made by subsequent withdrawal and reintroduction of the selected treatment regimen described herein.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A method of treating diseased or injured bone tissue comprising:
selecting an interior area in a bone tissue to be treated;
inserting a device into the interior area of the bone tissue to be treated; and
internally supporting the bone tissue using the device during treatment.

2. The method according to claim 1, further comprising selecting the interior area using a minimally invasive image-guided technique.

3. The method according to claim 1, wherein the device is replaceable.

4. The method according to claim 3, wherein the device is a catheter having a structure capable of supporting the bone tissue.

5. The method according to claim 4, wherein the catheter comprises an expandable three-dimensional, semi-compliant structure and fastener, and wherein the fastener releasably connects the catheter to the semi-compliant structure.

6. The method according to claim 5, wherein the catheter is detachable from the semi-compliant structure.

7. The method according to claim 6, wherein the catheter has a main body defining at least one interior passage therethrough, the semi-compliant structure defines an interior space, and the semi-compliant structure comprises a sealable port that allows for communication between the interior passage of the catheter and the interior space of the semi-compliant structure.

8. The method according to claim 7, further comprising injecting a bone supporting material into the semi-compliant structure through the passage within the catheter, through the sealable port, and into the interior space of the structure, thereby expanding the three-dimensional semi-compliant structure within the bone tissue.

9. The method according to claim 8, wherein the bone supporting material is capable of hardening to provide permanent bone support for the bone tissue.

10. The method according to claim 8, further comprising:
admixing a radiological treatment material with the bone supporting material to form an admixture and introducing the admixture into the semi-compliant structure, wherein the semi-compliant structure is capable of facilitating exposure of the area to be treated to the radiological material; and
exposing the bone tissue to the radiological treatment.

11. The method according to claim 10, further comprising replacing the semi-compliant structure with a second, replacement three-dimensional semi-compliant structure when the radiological treatment is depleted.

12. The method according to claim 10, wherein the radiological treatment is a time-release medication, and the medication may exfiltrate the semi-compliant structure and coat the surface of the semi-compliant structure.

13. The method according to claim 10, wherein the semi-compliant structure comprises a semi-permeable material and the medication diffuses through the semi-permeable material to treat a disease or injury.

14. The method according to claim 13, wherein the semi-permeable material is selected from the group consisting of polymeric materials, resorbable synthetic material, suture material, Nitinol, and combinations thereof.

15. The method according to claim 14, wherein the material is a biocompatible Nitinol.

16. The method according to claim 14, wherein the polymeric material is selected from the group consisting of polyesters, polyethylenes, polylactic acids, and combinations thereof.

17. The method according to claim 13, wherein the disease or injury is selected from the group consisting of osteoporosis, osteoporotic fractured metaphyseal and epiphyseal bone, osteoporotic vertebral bodies, fractures of vertebral bodies due to tumors, round cell tumors, avascular necrosis of the epiphyses of long bones, avascular necrosis of the proximal femur, distal femur and/or proximal humerus, defects arising from endocrine conditions, metastatic tumors, and combinations thereof.

18. The method according to claim 8, wherein the semi-compliant structure is detachable from the catheter, and the method further comprises:
detaching the catheter from the semi-compliant structure;
sealing the sealable port; and
maintaining the bone supporting material within the structure in a pressurized environment, thereby preventing the bone supporting material from exuding from within the structure, to provide temporary support of the bone tissue.

19. The method according to claim 8, wherein the bone supporting material is selected from the group consisting of a liquid, paste, gel, cement, and combinations thereof.

20. The method according to claim 19, wherein the bone supporting material is a cement.

21. The method according to claim 20, wherein the cement comprises a material selected from the group consisting of polymethylmethacrylate, dextran, polyethylene, carbon fiber, polyvinyl alcohol, and poly(ethylene terephthalate).

22. The method according to claim 19, wherein the bone supporting material is a liquid.

23. The method according to claim 18, further comprising:
reattaching the catheter to the sealable port; and
withdrawing the bone supporting material.

24. A device for treating diseased or injured bone comprising:
a catheter, wherein the catheter comprises a main body defining at least one interior passage therethrough;
an expandable semi-compliant structure, wherein the semi-compliant structure defines an interior space; and
a removable fastener, wherein the fastener removably connects the catheter to the semi-compliant structure.

25. The device according to claim 24, further comprising a sealable port, through which the interior passage of the catheter communicates with the interior space of the semi-compliant structure.

26. The device according to claim 25, wherein the catheter is detachable from the semi-compliant structure.

27. The device according to claim 26, wherein the catheter is capable of receiving bone supporting material.

28. The device according to claim 24, wherein the fastener is a screw device.

29. The device according to claim 24, wherein the semi-compliant structure is capable of expanding to adapt an area to be treated in a bone.
